SEQUENCE LISTING (12 sequences)

SEQ ID NO: 1 - protein, 1270 aa, Homo sapiens
  MISC_FEATURE (1)..(409): acidic N-terminal region
MTDDNSDDKIEDELQTFFTSDKDGNTHAYNPKSPPTQNSSASSVNWNSANPDDMVVDYETDPAVVTGENISLSLQGVEVFGHEKSSSDFISKQVLDMHKDSICQCPALVGTEKPKYLQHSCHSLEAVEGQSVEPSLPFVWKPNDNLNCAGYCDALELNQTFDMTVDKVNCTFISHHAIGKSQSFHTAGSLPPTGRRSGSTSSLSYSTWTSSHSDKTHARETTYDRESFENPQVTPSEAQDMTYTAFSDVVMQSEVFVSDIGNQCACSSGKVTSEYTDGSQQRLVGEKETQALTPVSDGMEVPNDSALQEFFCLSHDESNSEPHSQSSYRHKEMGQNLRETVSYCLIDDECPLMVPAFDKSEAQVLNPEHKVTETEDTQMVSKGKDLGTQNHTSELILSSPPGQKVGSSFGLTWDANDMVISTDKTMCMSTPVLEPTKVTFSVSPIEATEKCKKVEKGNRGLKNIPDSKEAPVNLCKPSLGKSTIKTNTPIGCKVRKTEIISYPRPNFKNVKAKVMSRAVLQPKDAALSKVTPRPQQTSASSPSSVNSRQQTVLSRTPRSDLNADKKAEILINKTHKQQFNKLITSQAVHVTTHSKNASHRVPRTTSAVKSNQEDVDKASSSNSACETGSVSALFQKIKGILPVKMESAECLEMTYVPNIDRISPEKKGEKENGTSMEKQELKQEIMNETFEYGSLFLGSASKTTTTSGRNISKPDSCGLRQIAAPKAKVGPPVSCLRRNSDNRNPSADRAVSPQRIRRVSSSGKPTSLKTAQSSWVNLPRPLPKSKASLKSPALRRTGSTPSIASTHSELSTYSNNSGNAAVIKYEEKPPKPAFQNGSSGSFYLKPLVSRAHVHLMKTPPKGPSRKNLFTALNAVEKSRQKNPRSLCIQPQTAPDALPPEKTLELTQYKTKCENQSGFILQLKQLLACGNTKFEALTVVIQHLLSEREEALKQHKTLSQELVNLRGELVTASTTCEKLEKARNELQTVYEAFVQQHQAEKTERENRLKEFYTREYEKLRDTYIEEAEKYKMQLQEQFDNLNAAHETSKLEIEASHSEKLELLKKAYEASLSEIKKGHEIEKKSLEDLLSEKQESLEKQINDLKSENDALNEKLKSEEQKRRAREKANLKNPQIMYLEQELESLKAVLEIKNEKLHQQDIKLMKMEKLVDNNTALVDKLKRFQQENEELKARMDKHMAISRQLSTEQAVLQESLEKESKVNKRLSMENEELLWKLHNGDLCSPKRSPTSSAIPLQSPRNSGSFPSPSISPR

SEQ ID NO: 2 - DNA, 32 bp, Artificial
  Synthetic oligonucleotide primer
ccgctcgaggactgacttgggatgcaaatgat

SEQ ID NO: 3 - DNA, 33 bp, Artificial
  Synthetic oligonucleotide primer
cggggtacctcatgcattaagagctgtaaataa

SEQ ID NO: 4 - RNA, 19 nt, Artificial
  Synthetic siRNA
uggcagagguuuaagguua

SEQ ID NO: 5 - protein, 16 aa, Drosophila melanogaster
RQIKIWFQNRRMKWKK

SEQ ID NO: 6 - protein, 1216 aa, Homo sapiens
  MISC_FEATURE (1)..(409): acidic N-terminal region
MTDDNSDDKIEDELQTFFTSDKDGNTHAYNPKSPPTQNSSASSVNWNSANPDDMVVDYETDPAVVTGENISLSLQGVEVFGHEKSSSDFISKQVLDMHKDSICQCPALVGTEKPKYLQHSCHSLEAVEGQSVEPSLPFVWKPNDNLNCAGYCDALELNQTFDMTVDKVNCTFISHHAIGKSQSFHTAGSLPPTGRRSGSTSSLSYSTWTSSHSDKTHARETTYDRESFENPQVTPSEAQDMTYTAFSDVVMQSEVFVSDIGNQCACSSGKVTSEYTDGSQQRLVGEKETQALTPVSDGMEVPNDSALQEFFCLSHDESNSEPHSQSSYRHKEMGQNLRETVSYCLIDDECPLMVPAFDKSEAQVLNPEHKVTETEDTQMVSKGKDLGTQNHTSELILSSPPGQKVGSSFGLTWDANDMVISTDKTMCMSTPVLEPTKVTFSVSPIEATEKCKKVEKGNRGLKNIPDSKEAPVNLCKPSLGKSTIKTNTPIGCKVRKTEIISYPRPNFKNVKAKVMSRAVLQPKDAALSKVTPRPQQTSASSPSSVNSRQQTVLSRTPRSDLNADKKAEILINKTHKQQFNKLITSQAVHVTTHSKNASHRVPRTTSAVKSNQEDVDKASSSNSACETGSVSALFQKIKGILPVKMESAECLEMTYVPNIDRISPEKKGEKENGTSMEKQELKQEIMNETFEYGSLFLGSASKTTTTSGRNISKPDSCGLRQIAAPKAKVGPPVSCLRRNSDNRNPSADRAVSPQRIRRVSSSAGNAAVIKYEEKPPKPAFQNGSSGSFYLKPLVSRAHVHLMKTPPKGPSRKNLFTALNAVEKSRQKNPRSLCIQPQTAPDALPPEKTLELTQYKTKCENQSGFILQLKQLLACGNTKFEALTVVIQHLLSEREEALKQHKTLSQELVNLRGELVTASTTCEKLEKARNELQTVYEAFVQQHQAEKTERENRLKEFYTREYEKLRDTYIEEAEKYKMQLQEQFDNLNAAHETSKLEIEASHSEKLELLKKAYEASLSEIKKGHEIEKKSLEDLLSEKQESLEKQINDLKSENDALNEKLKSEEQKRRAREKANLKNPQIMYLEQELESLKAVLEIKNEKLHQQDIKLMKMEKLVDNNTALVDKLKRFQQENEELKARMDKHMAISRQLSTEQAVLQESLEKESKVNKRLSMENEELLWKLHNGDLCSPKRSPTSSAIPLQSPRNSGSFPSPSISPR

SEQ ID NO: 7 - DNA, 32 bp, Artificial; identical to SEQ ID NO: 2
  Synthetic oligonucleotide Primer

SEQ ID NO: 8 - DNA, 36 bp, Artificial
  Synthetic oligonucleotide Primer
cggggtacctcaaaacaacgcagaaacggacccggt

SEQ ID NO: 9 - DNA, 35 bp, Artificial
  Synthetic oligonucleotide Primer
ccgctcgagcatatggttctctgtttttgggctct

SEQ ID NO: 10 - DNA, 33 bp, Artificial; identical to SEQ ID NO: 3
  Synthetic oligonucleotide Primer

SEQ ID NO: 11 - DNA, 32 bp, Artificial
  Synthetic oligonucleotide Primer
ccgctcgaggatctggtaatgccgctgtcatc

SEQ ID NO: 12 - DNA, 33 bp, Artificial; identical to SEQ ID NO: 3
  Synthetic oligonucleotide Primer

(19) United States
(12) Patent Application Publication
(10) Pub. No.: US 2013/0345145 A1
(43) Pub. Date: Dec. 26, 2013

(54) ATIP3 AND BIOLOGICALLY ACTIVE FRAGMENTS THEREOF FOR USE IN THE TREATMENT OF CANCER

(71) Applicants: Clara Nahmias, Paris (FR); Sylvie Rodrigues-Ferreira, Chevry-Cossigny (FR)

(72) Inventors: Clara Nahmias, Paris (FR); Sylvie Rodrigues-Ferreira, Chevry-Cossigny (FR)

(73) Assignees: INSTITUT NATIONAL DE LA SANTE ET DE LA RECHERCHE MEDICALE (INSERM), Paris (FR); UNIVERSITE PARIS DESCARTES, Paris (FR); CENTRE NATIONAL DE LA RECHERCHE SCIENTIFIQUE (C.N.R.S), Paris (FR)

(21) Appl. No.: 14/003,635

(22) PCT Filed: Mar. 12, 2012
(86) PCT No.: PCT/EP12/54291

§ 371 Date: Sep. 6, 2013

(30) Foreign Application Priority Data
Mar. 10, 2011 (EP) 11305262.5

(51) Int. Cl. C07K 14/47; A61K 31/337; A61K 38/17; C07K 16/18

(52) U.S. Cl. CPC C07K 14/47; C07K 16/18; A61K 31/337; A61K 38/1709
USPC 514/19.4; 530/350; 530/387.9; 514/19.3; 514/19.5; 514/19.8; 435/7.23; 435/6.12

(57) ABSTRACT

The present invention concerns a combination of (i) a polypeptide comprising ATIP3 or a biologically active fragment thereof, and (ii) a chemotherapeutic drug that is an antimitotic agent, for simultaneous or sequential use in the treatment of a patient suffering from cancer, e.g. a triple-negative breast cancer. The present invention also relates to a polypeptide comprising ATIP3 or a biologically active fragment thereof, for use in sustaining drug effect, increasing or restoring or enhancing sensitivity of a patient suffering from cancer to a chemotherapeutic drug that is an anti-mitotic agent. The present invention further provides biologically active polypeptides that comprise or consist of fragments of ATIP3, and antibodies binding thereto.

The present invention concerns a combination of (i) a polypeptide comprising ATIP3 or a biologically active fragment thereof, and (ii) a chemotherapeutic drug that is an anti-mitotic agent, for simultaneous or sequential use in the treatment of a patient suffering from cancer, e.g. a triple-negative breast cancer. The present invention also relates to a polypeptide comprising ATIP3 or a biologically active fragment thereof, for use in sustaining drug effect, increasing or restoring or enhancing sensitivity of a patient suffering from cancer to a chemotherapeutic drug that is an anti-mitotic agent. The present invention further provides biologically active polypeptides that comprise or consist of fragments of ATIP3, and antibodies binding thereto.

ATIP Proteins
Angiotensin-II type 2-receptor interacting proteins (ATIPs) are encoded by a single gene, the Microtubule-associated tumor suppressor 1 (MTUS1) gene. This gene comprises seventeen coding exons and three 5′ non-coding exons, scattered over more than 150 kb of genomic DNA at human chromosome 8p22. Five MTUS1 transcript variants can be classified into three major groups (ATIP1, ATIP3 and ATIP4) based on their tissue distribution and their utilization of 5′ exons. Thus, variants 1, 2 and 3 (ATIP3a, ATIP3b and ATIP2, respectively) all use the same 5′ coding exons (exons 1 and 2) and differ by alternative splicing of exons 3 and/or 4. Variants 4 (ATIP4) and 5 (ATIP1) use exons 5 and 8, respectively. The ATIP2 variant specifically uses exon 3, which contains a stop codon leading to premature truncation of the protein (see e.g. WO 01/57209, and, for a review, Rodrigues-Ferreira and Nahmias, 2010, Trends Endocrinol Metab. 21:684-90).

Rodrigues-Ferreira et al. (2009, PLoS One. 4(10):e7239 2009) have shown that ATIP3 is the major MTUS1 splice variant whose expression is significantly reduced in invasive breast tumors of high histological grade and triple-negative phenotype. Molecular cloning and functional studies revealed that ATIP3 is a novel mitotic spindle-associated protein that reduces breast cancer cell division in vitro and in vivo. In particular, ATIP3 was identified as a novel microtubule-associated protein whose expression is significantly reduced in highly proliferative breast carcinomas of poor clinical outcome. Since ATIP3 re-expression limits tumor cell proliferation in vitro and in vivo, Rodrigues-Ferreira et al. suggest that this protein may represent a novel useful biomarker and an interesting candidate for treatment of breast cancer.

Breast Cancer

Breast cancer remains the leading cause of death by malignancy in women worldwide. Its prevalence is as high as one woman among nine in western countries.

Over the past decade, traditional prognostic and predictive biomarkers including estrogen receptor (ER), progesterone receptor (PR) and human epidermal growth factor receptor 2 (HER2) have been routinely assessed by immunohistochemistry in newly diagnosed breast cancer (Dowsett and Dunbier, 2008, Clin Cancer Res. 14:8019-8026). Tumors that are positive for ER or PR are generally treated by hormonal therapy whereas patients with HER2 positive tumors are candidates for targeted anti-HER2 therapies. Tumors exhibiting a triple-negative phenotype (negative for ER, PR and HER2) currently have no targeted treatment available and remain of very poor prognosis (Rakha et al., 2008, J Clin Oncol. 26:2568-2581). Such triple-negative tumors represent 15% of total breast tumors, but as much 25% of deaths by this cancer.

The treatment of breast cancer, and in particular of breast tumors having a triple-negative phenotype, thus represents an issue of major public health importance. More specifically, there is a need in the art for the development of therapies for patients suffering from breast cancer, and in particular for targeted therapies for those patients suffering from triple-negative breast tumors.

### DESCRIPTION OF THE INVENTION

The inventors have surprisingly found that administration of ATIP3 sensitizes breast cancer cells to chemotherapy. In particular, expression of ATIP3 stabilizes microtubules and sensitizes breast cancer cells to lower doses of taxanes.

The inventors have thus found a new targeted therapy for treating cancer with no available targeted therapy. Said invention is particularly advantageous for treating triple-negative breast tumors. Without being bound to any theory, the inventors believe that re-expressing ATIP3 in such tumors, in which ATIP3 expression is significantly reduced, allows sustaining the effect of the drug, increasing or restoring sensitivity of tumor cells to therapeutic or prophylactic agents notably anti-mitotic agents such as taxanes. As a consequence, lower doses of anti-mitotic agents may be administered to the patient, thereby reducing side effects while maintaining efficacy of the anti-mitotic agent. In addition, ATIP3 has been shown to be a powerful anti-metastatic agent even in the absence of other chemotherapeutic drugs.

The inventors have further found a domain of ATIP3, consisting of residues 410 to 874, which exhibits the same anti-oncogenic activity as the full-length protein. This domain, which is much shorter than the full length protein, can thus advantageously be used for the treatment of cancer instead of full-length ATIP3. In addition, biologically active peptides can be derived therefrom.

ATIP3 and Biologically Active Fragments Thereof.

The polypeptides according to the invention, for use in the treatment of cancer as further described herein, are polypeptides comprising or consisting of ATIP3 or a biologically active fragment thereof.

As used throughout the present specification, the term “ATIPs” refers to Angiotensin-II type 2-receptor interacting proteins also called AT2-receptor interacting proteins. The term “ATIP3” refers both to the ATIP3a protein, also known under the name of microtubule-associated tumor suppressor 1 isoform 1 (see e.g. NCBI Reference Sequence: NP_{—}001001924 and UniProtKB/Swiss-Prot entry No. Q9ULD2-1, version 64 dated Jan. 11, 2011), and to the ATIP3b protein, also known under the name of microtubule-associated tumor suppressor 1 isoform 2 (see e.g. NCBI Reference Sequence: NP_{—}001001925 and UniProtKB/Swiss-Prot entry No. Q9ULD2-2, version 64 dated Jan. 11, 2011). Isoforms 1 and 2 of the Microtubule-associated tumor suppressor 1 are encoded by the MTUS1 gene. More specifically, these isoforms comprise the amino acids encoded by exons 1 and 2 of the human MTUS1 gene, or of the corresponding exons in another species than Homo sapiens, but not the amino acids encoded by exon 3 (see Rodrigues-Ferreira and Nahmias, 2010, Trends Endocrinol Metab. 21:684-90, and in particular FIG. 1 at page 685).

According to the invention, ATIP3 preferably is a human ATIP3.

ATIP3 may for example have the sequence of SEQ ID NO: 1 (human ATIP3a) or SEQ ID NO: 6 (human ATIP3b), or correspond to a variant thereof (preferably an allelic variant), or to a homologous protein thereof in another species than Homo sapiens.

Most preferably, ATIP3 has a sequence consisting of:
the sequence of SEQ ID NO: 1,
the sequence of SEQ ID NO: 6, or
a sequence at least 80%, 85%, 90%, 95%, 96%, 97%, 98% or 99% identical to the sequence SEQ ID NO: 1 or SEQ ID NO: 6, provided the encoded polypeptide is biologically active. Such a sequence may for example correspond to an allelic variant of human ATIP3 proteins of SEQ ID NO: 1 or SEQ ID NO: 6.

Allelic variants of human ATIP3 proteins of SEQ ID NO: 1 or 6 are known to the skilled in the art. For instance, allelic variants of a human ATIP3 protein of SEQ ID NO: 1 or of a human ATIP3 protein of SEQ ID NO: 6 are disclosed in UniProtKB/Swiss-Prot entry No. Q9ULD2, version 64 dated Jan. 11, 2011.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e., the two sequences are compared over their entire length). Methods for comparing the identity and homology of two or more sequences are well known in the art. The <<needle>> program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Polypeptides consisting of an amino acid sequence “at least 80%, 85%, 90%, 95%, 96%, 97%, 98% or 99% identical” to a reference sequence may comprise mutations such as deletions, insertions and/or substitutions compared to the reference sequence. The polypeptide consisting of an amino acid sequence at least 80%, 85%, 90%, 95%, 96%, 97%, 98% or 99% identical to a reference sequence may correspond to an allelic variant of the reference sequence. It may for example only comprise substitutions compared to the reference sequence. The substitutions preferably correspond to conservative substitutions as indicated in the table below.

| Conservative substitutions | Type of Amino Acid |
|---|---|
| Ala, Val, Leu, Ile, Met, Pro, Phe, | Amino acids with aliphatic |
| Trp | hydrophobic side chains |
| Ser, Tyr, Asn, Gln, Cys | Amino acids with uncharged but |
| | polar side chains |
| Asp, Glu | Amino acids with acidic side chains |
| Lys, Arg, His | Amino acids with basic side chains |
| Gly | Neutral side chain |

Also preferably, the substitutions may correspond to polymorphic variations such as, e.g. those described in UniProtKB/Swiss-Prot entry No. Q9ULD2, version 64 dated Jan. 11, 2011 and/or those described in the dbSNP database that is available on the ncbi.nlm.nih.gov/projects/SNP/ website. For example, the ATIP3 polypeptide according to the invention may comprise one or more substitutions selected from the group consisting of rs61733703, rs61742544, rs61733702, rs3739407, rs61733701, rs3739408, rs61733698, rs73206291, rs61733697, rs209568, rs113749634, rs61733696, rs11203910, rs61733695, rs41528945, rs75253845, rs61733694, rs17690844, rs61740538, rs2979788, rs209569, s77588184, rs113445081, s61733691, rs75653293, rs75383511, rs61733690, rs2979792, rs74754090, rs61748836, rs112428784, rs71882531, rs72007854, rs71838742, rs72185938, s72175917, rs61733708, rs34504210, rs34069077, rs12224, rs35188785, s61733707, rs34178284, rs61999336, rs72190379, rs17853231, rs71990301, rs111237660, rs61733705, rs76290497, rs61733704 and rs12550193.

The polypeptides according to the invention, for use in the treatment of cancer as further described herein, not only encompass polypeptides comprising or consisting of full-length ATIP3, but also polypeptides comprising or consisting of fragments thereof, provided the fragments are biologically active.

In the frame of the present invention, the biologically active fragment may for example comprise at least 6, 8, 10, 12, 15, 18, 20, 25, 30, 50, 75, 100, 125, 150, 200, 250, 275, 300, 325, 350, 375, 400, 425, 450, 460, 464, 475, 500, 550, 600, 700, 800, 900, 1000 or 1110 consecutive amino acids of ATIP3.

By “biological activity” of ATIP3 or a fragment thereof is meant (i) the capacity to reduce tumor growth (i.e. it presents an anti-oncogenic activity); and/or (ii) the capacity to reduce tumor metastasis; and/or (iii) the capacity of associating with microtubules; and/or (iv) the capacity to promote prolonged mitosis (iv) the capacity of sustaining drug effect, and/or (v) the capacity of increasing or restoring sensitivity of cancer cells to therapeutic or prophylactic agents.

The skilled in the art can easily determine whether ATIP3 or a fragment thereof is biologically active. For example, the capacity to reduce tumor growth can for example be determined by assessing breast cancer cell proliferation through clonogenicity experiments (e.g. as described in Example 2) and/or assessing tumor growth in vivo (e.g. using the protocol provided in Rodrigues-Ferreira et al., 2009, PLoS One. 4(10):e7239 2009). The capacity to reduce tumor metastasis can for example be determined by assessing breast cancer cell migration through boyden chambers or wound healing experiments (e.g. as described in Example 4) and/or assessing tumor metastasis in vivo (e.g. as described in the example provided herein). The capacity of associating with microtubules can be determined by analyzing co-localization with alpha-tubulin in immunofluorescence studies and/or assessing microtubule binding in cell extract, e.g. using the co-sedimentation protocol described in Rodrigues-Ferreira et al. (2009, PLoS One. 4(10):e7239 2009). The capacity to promote prolonged mitosis can be determined by performing live cell imaging of cells expressing mCherry-histone 2B and transfected with the polypeptide to be tested fused to GFP, e.g. using the protocol described in Rodrigues-Ferreira et al. (2009, PLoS One. 4(10):e7239 2009).

The biological activity of ATIP3 may also be determined by assessing the capacity of ATIP3 to bind to its natural binding partners such as e.g. EB1, AT2 receptor and tubulin. The binding of ATIP3 to its natural binding partners may for example be assessed using a co-immunoprecipitation assay, a pull-down assay or the yeast two hybrid system (Y2H).

The activity of ATIP3 may further be assessed by determining if ATIP3 peptide sequence and/or ATIP3 gene is downregulated using methods which are well known to the person skilled in the art, including in particular immunologic methods such as detection using polyclonal or monoclonal antibodies or methods involving PCR or reverse transcriptase PCR(RT-PCR), methods involving the use of DNA arrays (macroarrays or microarrays) and In Situ hybridizations.

As used herein, a “biologically active” fragment refers to a fragment exhibiting at least one, preferably all, of the biological activities of a full-length ATIP3 polypeptide of SEQ ID NO: 1, provided the biologically active fragment retains the capacity of reducing tumor growth. The biologically active fragment may for example be characterized in that it is capable of reducing breast cancer cell proliferation when assessed through clonogenicity experiments (see Example 2 and FIG. 7) and/or reducing cell migration when assessed through Boyden chambers or wound healing experiments (see Example 4 and FIGS. 4 and 5)

The inventors have identified a functional domain of ATIP3 of about 465 amino acids that retains the anti-oncogenic activity of full-length ATIP3. They have further found two consensus sites for interaction with the EB1 protein (see Example 2 and FIG. 6).

Therefore, the present invention provides a polypeptide comprising or consisting of a biologically active fragment of at most 500 consecutive amino acids of ATIP3, wherein said fragment comprises or consists of:
a) the sequence consisting of amino acids 410 to 874 of SEQ ID NO: 1;
b) the sequence consisting of amino acids 410 to 820 of SEQ ID NO: 6;
c) the sequence consisting of amino acids 333 to 580 of SEQ ID NO: 1;
d) a sequence at least 80, 85, 90, 95, 96, 97, 98 or 99% identical to the sequence of (a), (b) or (c);
e) at least 6, 8, 10, 12, 15, 18, 20, 30, 25, 50, 75, 100, 125, 150, 200, 250, 275, 300, 325, 350, 375, 400, 425, 450 or 460 consecutive amino acids of the sequence of (a), (b), (c) or (d);
f) the sequence of (d) or (e), wherein said sequence comprises the KNIP EB1-binding sequence consisting of amino acids 462 to 465 of SEQ ID NO: 1 or SEQ ID NO: 6; or amino acids 410 to 634 or 705 to 874 or 817 to 874 of SEQ ID NO: 1,
g) the sequence consisting of amino acids 410 to 634 or 705 to 874 or 817 to 874 of SEQ ID NO: 1,
h) a sequence at least 80, 85, 90, 95, 96, 97, 98 or 99% identical to the sequence of (g),
i) at least 6, 8, 10, 12, 15, 18, 20, 25, 50, 75, 90, 100, 120, 125, 130, 135, 140, 145, 150, 155 or 160 consecutive amino acids of the sequence of (g) or (h);
j) the sequence consisting of amino acids 1240 to 1270 of SEQ ID NO: 1;
k) a sequence at least 80, 85, 90, 95, 96, 97, 98 or 99% identical to the sequence of (j);
l) at least 6, 8, 10, 12, 15, 18, 20 or 25 consecutive amino acids of the sequence of (j) or (k); or
m) the sequence of (k) or (l), wherein said sequence comprises the SAIP EB1-binding sequence consisting of amino acids 1249 to 1252 of SEQ ID NO: 1.

According to the invention, a polypeptide comprising or consisting of a biologically active fragment of at most 500 consecutive amino acids of ATIP3 does not contain more than 500 consecutive amino acids of ATIP3. Indeed, “a polypeptide comprising or consisting of a biologically active fragment of at most 500 consecutive amino acids of ATIP3” according to the invention refers to a polypeptide of at most 500 consecutive amino acids of ATIP3. Consequently, said “polypeptide comprising or consisting of a biologically active fragment of at most 500 consecutive amino acids of ATIP3” is not ATIP3 protein.

Nonetheless, a polypeptide comprises ATIP3 or a biologically active fragment thereof is envisaged by the invention in combination with a chemotherapeutic drug for simultaneous or sequential use in the treatment of a patient suffering from cancer.

In one embodiment, said polypeptide comprising or consisting of a biologically active fragment of at most 500 consecutive amino acids of ATIP3 is characterized in that it comprises the KNIP EB1-binding sequence (amino acids 462 to 465 of SEQ ID NO: 1).

In another embodiment, said polypeptide comprising or consisting of a biologically active fragment of at most 500 consecutive amino acids of ATIP3 is characterized in that it comprises the SAIP EB1-binding sequence (amino acids 1249 to 1252 of SEQ ID NO: 1).

The above polypeptides comprising or consisting of a biologically active fragment of at most 500 consecutive amino acids of ATIP3 can advantageously be used as medicaments. The invention thus pertains to a pharmaceutical composition comprising such a polypeptide and a pharmaceutically acceptable carrier. In particular, such polypeptides can be used for treating cancer (said cancer being defined in detail herebelow), either alone or in combination with a chemotherapeutic drug such as an anti-mitotic agent (as further described herebelow).

In the frame of the present invention, the above polypeptides comprising or consisting of a biologically active fragment of at most 500 consecutive amino acids of ATIP3 are preferred polypeptides comprising or consisting of ATIP3 or a biologically active fragment of ATIP3.

In a specific embodiment, the above polypeptide comprising or consisting of a biologically active fragment of at most 500 consecutive amino acids of ATIP3 is a peptide.

In further specific embodiments, said polypeptide comprises or consists of a biologically active fragment of at most 500 consecutive amino acids of ATIP3 with the provisio that said polypeptide does not consist of amino acids 856 to 876, 54 to 685 or 686 to 815 of sequence SEQ ID NO: 1. Contrary to the polypeptides according to the invention, the polypeptides consisting of amino acids 856 to 876, 54 to 685 or 686 to 815 of sequence SEQ ID NO: 1 don't bind to microtubules or EB1 protein.

As used herein, the term “polypeptide” refers to a chain of at least six amino acids, preferably linked by peptide bonds. This term encompasses, e.g., proteins and peptides.

The term “peptide” refers to a polypeptide that is at most 50 amino acids long, preferably at most 30 amino acid long. The peptide may optionally be chemically modified, e.g. in order to enhance its bioactivity and/or its biodisponibility. For example, the peptide may comprise one or more of the following chemical modifications:
replacement(s) of an amino acid with a modified and/or unusual amino acid, e.g. a replacement of an amino acid with an unusual amino acid like Nle, Nva or Orn; and/or
replacement(s) of an amino acid with an amino acid having a different chirality; and/or
modification(s) to the N-terminal and/or C-terminal ends of the peptides such as e.g. N-terminal acylation (preferably acetylation) or desamination, or modification of the C-terminal carboxyl group into an amide or an alcohol group; and/or
modification(s) at the amide bond between two amino acids: acylation (preferably acetylation) or alkylation (preferably methylation) at the nitrogen atom or the alpha carbon of the amide bond linking two amino acids; and/or
modification(s) at the alpha carbon of the amide bond linking two amino acids such as e.g. acylation (preferably acetylation) or alkylation (preferably methylation) at the alpha carbon of the amide bond linking two amino acids; and/or
modification(s) allowing the obtention of azapeptide(s), in which one or more alpha carbons are replaced with nitrogen atoms; and/or
modification(s) allowing the obtention of betapeptide(s), in which the amino group of one or more amino acid is bonded to the β carbon rather than the a carbon.

In addition to ATIP3 or to the biologically active fragment thereof (in particular to the biologically active fragment of at most 500 consecutive amino acids of ATIP3 as defined hereabove), the polypeptides according to the invention may comprise further amino acids sequences such as e.g. a signal peptide and/or a pro-peptide (in order to allow expression of the polypeptide in a host cell), a tag useful for purification (e.g. a His-tag, which typically consists of six histidine residues), and/or a sequence useful for transducing the polypeptide inside eukaryotic cells (e.g. penetratin).

In a specific embodiment, the polypeptide according to the invention comprises penetratin. Penetratin is a 16 amino acids-long peptide, the sequence of which is shown as SEQ ID NO: 5, that is derived from antennapedia homeodomain. This peptide is positively charged and capable of crossing cell membranes through transduction. It can thus be used for transduction of molecular cargoes inside eukaryotic cells. It is both efficient in vitro and in vivo (Prochiantz, 2008, Adv Drug Deliv Rev. 60:448-51). Preferably, penetratin is fused to the C-terminal extremity of ATIP3 or of the biologically active fragment thereof.

The polypeptide according to the invention may further comprise a His-tag fused to the N-terminal extremity of ATIP3 or of the biologically active fragment thereof, and optionally a signal peptide fused to the N-terminal extremity of the His-tag.

The polypeptides according to the invention may be produced using any method known in the art. They may for example be produced as recombinant polypeptides in a host cell (e.g. in a bacterial, yeast or eukaryotic host cell), or chemically synthesized (e.g. when the polypeptide is a peptide).

The invention also provides nucleic acids encoding and/or expressing ATIP3 or a biologically active fragment thereof, in particular expression vectors encoding and/or expressing ATIP3 or a biologically active fragment thereof. Such nucleic acids find for example use when ATIP3 or a biologically active fragment thereof is intended to be administered to a patient using an adenoviral vector, and/or in the frame of a gene therapy.

Combination of ATIP3 or of a Biologically Active Fragment Thereof with a Chemotherapeutic Drug, for Use in the Treatment of Cancer

The inventors have found that a polypeptide comprising or consisting of ATIP3 or a biologically active fragment thereof sustains, increases or restores sensitivity of cancer cells to therapeutic or prophylactic agents and notably prevents taxol or docetaxel reduction of effectiveness and sensitizes breast cancer cells to the taxol and docetaxel anti-mitotic agents. Therefore, the present invention provides a combination of
i. a polypeptide comprising or consisting of ATIP3 or a biologically active fragment thereof (as defined in the above paragraph), or a nucleic acid coding therefore; and
ii. a chemotherapeutic drug that is an anti-mitotic agent;
   for simultaneous or sequential use in the treatment of a patient suffering from cancer, notably in the treatment of a patient suffering from an ATIP3 under-expressing cancer.

The present invention also provides a polypeptide comprising or consisting of ATIP3 or a biologically active fragment thereof (as defined in the above paragraph), for use treating ATIP3 under-expressing cancer, in sustaining drug effect or in increasing, restoring or enhancing sensitivity of a patient suffering from cancer to a chemotherapeutic drug that is an anti-mitotic agent.

“Sustaining drug effect” refers to a circumstance when a disease (e.g., cancer) responds with less efficiency or ceases responding to the therapeutic agent or agents to which the disease had previously been responsive. For cancers, such therapeutic agent may be a chemotherapeutic drug such as anthracyclin, vinca alkaloids, topoisomerase inhibitors, taxanes, or other small molecules used in cancer chemotherapy.

By a “chemotherapeutic drug” is meant a drug that has a marketing approval for the treatment of cancer, or a drug undergoing clinical or preclinical trial for the treatment of cancer.

By an “anti-mitotic agent”, also referred to as a “spindle poison” or a “mitosis poison”, is meant an agent that is capable of slowing down and/or inhibiting mitosis. Such anti-mitotic agents can for example stabilize tubulin and thus “freeze” the mitotic process (as in the case of most taxanes), or destroy mitotic spindles (as in the case of most vinca alkaloids).

In a preferred embodiment of the combination according to the invention, the chemotherapeutic drug is a taxane. The taxanes are diterpenes that were originally derived from plants of the genus Taxus (yews). Now, they are usually synthesized. Taxanes have been used to produce various chemotherapy drugs such as, e.g., Paclitaxel (Taxol), Docetaxel (Taxotere), Halaven (Eribulin) and Cabazitaxel. These taxanes, and especially Paclitaxel (Taxol), are preferred chemotherapeutic drugs that can be used in the frame of the present invention.

Alternatively, the chemotherapeutic drug can be a vinca alkaloid such as, e.g., vinblastine, vincristine, vindesine or vinorelbine.

The polypeptide comprising or consisting of ATIP3 or a biologically active fragment thereof and the chemotherapeutic drug may either be administered simultaneously to the patient, or sequentially. When the administration is sequential, the polypeptide comprising or consisting of ATIP3 or a biologically active fragment thereof is preferably administered prior to the chemotherapeutic drug in order to sensitize the patient.

In addition to the anti-mitotic agent and the polypeptide comprising ATIP3 or a biologically active fragment thereof, the treatment regimen of the patient may further comprise surgery, radiotherapy, hormone-therapy, immunotherapy, and/or administration of other chemotherapeutic drugs.

Since the polypeptide comprising ATIP3 or a biologically active fragment thereof sensitizes breast cancer cells to anti-mitotic agents, the anti-mitotic agents can advantageously be used at lower doses than in a treatment regimen wherein it is administered alone.

Therefore, in a preferred embodiment of the combination according to the invention, the chemotherapeutic drug is for use at a low dose, i.e. at a lower dose than the dose recommended when said drug is administered without said polypeptide comprising ATIP3 or a biologically active fragment thereof.

The skilled in the art can immediately determine a low dose for a given chemotherapeutic drug. Such a low dose notably depends on the cancer to be treated and on the therapeutic protocol.

In the frame of the present invention, by “low dose” is meant a dose that is inferior to the recommended dose that would be given to the patient when the chemotherapeutic drug is administered in the absence of the ATIP3 or a biologically active fragment thereof. Said low dose is preferably inferior of at least 10%, 15%, 20%, 25% or 50% to the recommended dose.

The recommended dose that would be given to the patient when the chemotherapeutic drug is administered in the absence of the ATIP3 or of the biologically active fragment thereof is known to the skilled in the art. Such a recommended dose can for example be found in the information provided by the authorities delivering marketing authorizations (e.g. in the EPARs published by the EMEA).

As an illustrative example, it will be described herebelow what is meant by a low dose of docetaxel.

For example, for the treatment of patients with locally advanced or metastatic breast cancer, the recommended dose of docetaxel is 100 mg/m² in monotherapy. Therefore, a low dose of docetaxel, in the frame of the treatment in monotherapy of patients with locally advanced or metastatic breast cancer, is a dose inferior to 100 mg/m², preferably inferior to 90 mg/m2, 75 mg/m² or 50 mg/m².

In contrast to this, when docetaxel is used as an adjuvant treatment of operable node-positive and node-negative breast cancer, the recommended dose of docetaxel is 75 mg/m² administered 1-hour after doxorubicin 50 mg/m² and cyclophosphamide 500 mg/m² every 3 weeks for 6 cycles (TAC regimen). More generally, docetaxel is usually administered at 75 mg/m² when associated with another drug (e.g. endoxan or capecitabin) or when the patient is at risk of not tolerating an aggressive chemotherapy. Therefore, a low dose of docetaxel, when associated with another drug or when the patient is believed not to tolerate an aggressive chemotherapy, is a dose inferior to 75 mg/m², preferably inferior to 50 mg/m², 40 mg/m² or 30 mg/m².

As another illustrative example, it will be described herebelow what is meant by a low dose of paclitaxel (Taxol).

Paclitaxel is usually administered at 80 or 90 mg/m² once a week (e.g. on day 1, 8, 15, and then on day 28 and each following week, optionally in combination with other drugs). Therefore, a low dose of paclitaxel is a dose inferior to 80 mg/m², preferably inferior to 70 mg/m², 60 mg/m², 50 mg/m² or 40 mg/m2.

In a preferred embodiment of the combination according to the invention, the polypeptide comprising or consisting of ATIP3 or a biologically active fragment invention is ATIP3.

In another preferred embodiment of the combination according to the invention, the polypeptide comprising or consisting of ATIP3 or a biologically active fragment thereof is a polypeptide comprising or consisting of a biologically active fragment of at most 500 consecutive amino acids of ATIP3, wherein said fragment comprises or consists of:
a) the sequence consisting of amino acids 410 to 874 of SEQ ID NO: 1;
b) the sequence consisting of amino acids 410 to 820 of SEQ ID NO: 6;
c) the sequence consisting of amino acids 333 to 580 of SEQ ID NO: 1;
d) a sequence at least 80, 85, 90, 95, 96, 97, 98 or 99% identical to the sequence of (a), (b) or (c);
e) at least 6, 8, 10, 12, 15, 18, 20, 25, 50, 75, 100, 125, 150, 200, 250, 275, 300, 325, 350, 375, 400, 425, 450, 460, 464, 475 consecutive amino acids of the sequence of (a), (b), (c) or (d);
f) the sequence of (d) or (e), wherein said sequence comprises amino acids 462 to 465 of SEQ ID NO: 1 or SEQ ID NO: 6; or amino acids 410 to 634 or 705 to 874 or 817 to 874 of SEQ ID NO: 1,
g) the sequence consisting of amino acids 410 to 634 or 705 to 874 or 817 to 874 of SEQ ID NO: 1,
h) a sequence at least 80, 85, 90, 95, 96, 97, 98 or 99% identical to the sequence of (g),
i) at least 6, 8, 10, 12, 15, 18, 20, 25, 50, 75, 90, 100, 120, 125, 130, 135, 140, 145, 150, 155 or 160 consecutive amino acids of the sequence of (g) or (h);
j) the sequence consisting of amino acids 1240 to 1270 of SEQ ID NO: 1;
k) a sequence at least 80, 85, 90, 95, 96, 97, 98 or 99% identical to the sequence of (j)
l) at least 6, 8, 10, 12, 15, 18, 20, or 25 consecutive amino acids of the sequence of (j) or (k); or
m) the sequence of (k) or (l), wherein said sequence comprises amino acids 1249 to 1252 of SEQ ID NO: 1.

The present invention further provides a methods of treating an individual in need thereof, said method comprising the step of administering an effective amount of:
a polypeptide comprising or consisting of ATIP3 or a biologically active fragment thereof, or a nucleic acid coding therefore; and
a chemotherapeutic drug that is an anti-mitotic agent;
   to an individual in need thereof.

The polypeptides and drugs are administered in an “effective amount”, i.e. in an amount sufficient to treat the cancer. It will be appreciated that this amount will vary with the effectiveness of therapeutic agent(s) employed, with the nature of any carrier used, with the seriousness of the disease and the age of the patient. The determination of appropriate amounts for any given composition is within the skill in the art, through standard series of tests designed to assess appropriate therapeutic levels.

By “individual in need thereof” is meant an individual suffering from cancer, or an individual that is in remission after having suffered from cancer.

In the frame of the present invention, the individual preferably is a human individual.

However, the veterinary use of the polypeptides and drugs according to the present invention is also envisioned. The individual may thus also correspond to a non-human individual, preferably a non-human mammal.

The term “treating” is meant to encompass both therapeutic and prophylactic methods, i.e. a method aiming at curing, improving the condition and/or extending the lifespan of an individual suffering from the cancer. It also refers to methods aiming at preventing the appearance or the spreading of metastases, as well as methods aiming at preventing a relapse.

Cancers to be Treated in Accordance with the Invention

The methods, combinations, uses and polypeptides according to the invention find use in the treatment of cancer.

As used throughout the present specification, the term “cancer” refers to any type of malignant (i.e. non benign) tumor. The malignant tumor may correspond to a primary tumor or to a secondary tumor (i.e. a metastasis). The tumor may correspond to a solid malignant tumor such as e.g. a carcinoma, an adenocarcinoma, a sarcoma, a melanoma, a mesothelioma or a blastoma.

The cancer preferably corresponds to a solid malignant tumor that is an epithelial cancer, e.g. a carcinoma or an adenocarcinoma involving malignant proliferation of epithelial tissue cells.

The cancer most preferably corresponds to a breast cancer (in particular a breast carcinoma), a pancreatic cancer, an ovary cancer, a head-and-neck cancer, a colon cancer, a colorectal cancer, a liver cancer (in particular a hepatocarcinoma), a stomach cancer, a prostate cancer, a bladder cancer or a Non-Small-Cell lung carcinoma. Indeed, these cancers are either cancers in which the MTUS1 gene is known to be sometimes under-expressed and/or mutated, and/or cancers that are commonly treated by administration of Taxol.

In particular, ATIP3 delays the time-course of metastatic progression and reduces the formation of cancer metastases. Therefore, the cancer to be treated in accordance with the present invention preferably corresponds to a cancer of high histological grade (i.e. a cancer which is believed to have a high proliferative potential) and/or a metastatic cancer.

The “histological grade” of a cancer is a well-known classification method of cancers, where cancers are classified from grade 1 to 4 (G1-4). More specifically, cancer cells are “low grade” if they appear similar to normal cells, and “high grade” if they appear poorly differentiated. The grade further takes into account the proliferative index of the cells. For example, a G3 or G4 cancer would be classified as a cancer of high histological grade.

By “metastatic cancer” is meant a cancer which has spread to at least one tissue or organ that is different from the tissue or organ in which the primary tumor is located.

In particular, it has been demonstrated that ATIP3 and biologically active fragments thereof allow reducing the tumor growth and the spread of metastasis in the case of breast cancer.

Therefore, in a preferred embodiment according to the invention, the cancer to be treated in accordance with the present invention is a breast cancer, most preferably a breast carcinoma.

In view of the highly efficient anti-oncogenic, and in particular anti-metastatic, effect of ATIP3, it is believed that the present invention is suitable for the treatment of triple-negative breast cancers. This hypothesis has been confirmed by the experimental data enclosed herein (see e.g. Example 3 and FIG. 11). This is all the more true when the polypeptide comprising or consisting of ATIP3 or a biologically active fragment invention is used in combination with an anti-mitotic agent, since those polypeptides are capable of sensitizing breast cancer cells to anti-mitotic agents, and thus allow the treatment of patients who had a very poor prognosis until now.

Therefore, in a most preferred embodiment according to the invention, the cancer is a triple-negative breast cancer and/or an ATIP3 under-expressing cancer.

By a “triple-negative breast cancer” is meant a breast tumor that does not express estrogen receptor (ER), progesterone receptor (PR) and epidermal growth factor receptor 2 (HER2). Determining whether a breast cancer is a triple-negative breast cancer is preferably carried out through immunohistochemistry (see, e.g., Dowsett and Dunbier, 2008, Clin Cancer Res. 14:8019-8026, and Rakha et al., 2008, J Clin Oncol. 26:2568-2581).

“Sensitizing cancer cells” to therapeutic or prophylactic agents means sustaining (over time), increasing or restoring sensitivity of cancer cells to said therapeutic or prophylactic agents, or both.

“ATIP3 under-expressing cancer” means cancers cells or a biological sample containing such cancer cells, expressing ATIP3 or a biologically active fragment thereof at a significantly lower level than in a control sample.

Selecting Patients for a Targeted Therapy

Since the polypeptide comprising ATIP3 or a biologically active fragment thereof are believed to be suitable for the treatment of triple-negative breast cancers, the present invention provides a targeted therapy for the treatment of patients suffering from such triple-negative breast cancers.

In particular, the present invention provides an in vitro method for selecting a patient to be treated by a polypeptide comprising ATIP3 or a biologically active fragment thereof (as defined hereabove), or by a nucleic acid coding therefore, optionally in combination with a chemotherapeutic drug that is an anti-mitotic agent (as defined hereabove), wherein said patient suffers from breast cancer, and wherein said method comprises the steps of:
a) providing or obtaining a biological sample comprising cells from said breast cancer;
b) determining whether said breast cancer is a triple-negative breast cancer, e.g. through immunochemistry; and
c) selecting said patient if he suffers from a triple-negative breast cancer.

Such a method can easily be routinely implemented since determining whether a breast cancer is a triple-negative breast cancer can be done using methods that are well-known and widely used in the art (see e.g. Dowsett and Dunbier, 2008, Clin Cancer Res. 14:8019-8026, and Rakha et al., 2008, J Clin Oncol. 26:2568-2581).

Also provided is a method of diagnosis and/or prognosis of a patient suffering from a low ATIP3-expressing cancer which method comprises the steps of:

a) providing a biological sample comprising cancer cells from said patient;
determining whether ATIP3 or a biologically active fragment thereof is expressed on said cancer cells; and

b) selecting said patient if said ATIP3 or a biologically active fragment thereof is expressed at a significantly lower level in said biological sample than in a control sample. Preferably, a said method is an in vitro or ex vivo method.

In one embodiment, said method comprises the step of determining whether the ATIP3 expressed is biologically active.

Protein expression may be assessed by using immunologic methods such as detection using polyclonal or monoclonal antibodies. Suitable immunologic methods include immunohistochemistry, tumor tissue-microarray hybridization and Western Blot.

Alterations in the levels of ATIP3 mRNA expression may be assessed by other methods which are well known to the person skilled in the art, including in particular quantitative methods involving reverse transcriptase PCR (RT-PCR), such as real-time quantitative RT-PCR (qRT-PCR), and methods involving the use of DNA arrays (macroarrays or microarrays) and In Situ hybridizations, or New Generation Sequencing (NGS).

ATIP3 or a biologically active fragment thereof are also suitable for the treatment of cancers in which ATIP3 is under-expressed, in order to increase or restore sensitivity to chemotherapeutic drugs or to sustain drug effect (see e.g. Example 3 and FIGS. 8 to 11).

Therefore, the present invention also provides an in vitro method for selecting a patient to be treated by a polypeptide comprising ATIP3 or a biologically active fragment thereof (as defined hereabove), or by a nucleic acid encoding such a polypeptide, preferably in combination with a chemotherapeutic drug that is an anti-mitotic agent (as defined hereabove), wherein said method comprises the steps of:
a) providing a biological sample comprising cells from said cancer;
b) determining whether ATIP3 is expressed in said biological sample; and
c) selecting said patient if ATIP3 is expressed at a significantly lower level in said biological sample than in a control sample.

The control sample may e.g. correspond to a tissue sample from a healthy individual (preferably from the same tissue as the cancer cells), to a sample of healthy tissue from the patient (e.g. healthy tissue surrounding the cancerous tissue), or to a sample comprising an amount of ATIP3 that is indicative of the expression level of ATIP3 in a healthy individual.

Determining whether ATIP3 is expressed can be done using methods well-known in the art, e.g. through immunochemistry, western blot or RT-qPCR. ATIP3 expression may be assessed at the protein level or at the mRNA level.

As used herein, an expression level is “significantly” lower when a difference can be detected using conventional methods (e.g. through immunochemistry, western blot or RT-qPCR). Preferably, the patient is selected if expression of ATIP3 exhibits a reduction of at least 2-fold in said biological sample compared to the control sample.

Pharmaceutical Compositions According to the Invention

The present invention also provides a pharmaceutical composition comprising:
i. ATIP3 or a biologically active fragment thereof (as defined hereabove), or a nucleic acid coding therefore,
ii. a pharmaceutically acceptable carrier; and
iii. optionally a chemotherapeutic drug that is an anti-mitotic agent (as defined hereabove.

Pharmaceutical compositions formulated in a manner suitable for administration to human are known to the skilled in the art. The pharmaceutical composition of the invention may further comprise stabilizers, buffers, etc.

The compositions of the present invention may for example be formulated and used as tablets, capsules or elixirs for oral administration, suppositories for rectal administration, sterile solutions or suspensions for injectable administration.

The choice of the formulation ultimately depends on the intended way of administration, such as e.g. an intravenous, intraperitoneal, subcutaneous or oral way of administration, or a local administration via tumor injection.

The pharmaceutical composition according to the invention may be a solution or suspension, e.g. an injectable solution or suspension. It may for example be packaged in dosage unit form.

ATIP3 or a biologically active fragment thereof can be administered as a polypeptide or a peptide (e.g. formulated in a liposome or in nanoparticles), or as a nucleic acid therefore (e.g. under the form of an adenoviral vector, for example formulated in a liposome).

Antibodies According to the Invention

The invention further provides an antibody that specifically binds to a polypeptide comprising or consisting of a biologically active fragment of at most 500 consecutive amino acids of ATIP3, wherein said fragment comprises or consists of:
a) the sequence consisting of amino acids 410 to 874 of SEQ ID NO: 1;
b) the sequence consisting of amino acids 410 to 820 of SEQ ID NO: 6;
c) the sequence consisting of amino acids 333 to 580 of SEQ ID NO: 1;
d) a sequence at least 80, 95, 90, 95, 96, 97, 98 or 99% identical to the sequence of (a), (b) or (c);
e) at least 6, 8, 10, 12, 15, 18, 20, 25, 50, 75, 100, 125, 150, 200, 250, 275, 300, 325, 350, 375, 400, 425, 450, 460, 464, 475 consecutive amino acids of the sequence of (a), (b), (c) or (d);
f) the sequence of (d) or (e), wherein said sequence comprises amino acids 462 to 465 of SEQ ID NO: 1 or SEQ ID NO: 6; or amino acids 410 to 634 or 705 to 874 or 817 to 874 of SEQ ID NO: 1
g) the sequence consisting of amino acids 410 to 634 or 705 to 874 or 817 to 874 of SEQ ID NO: 1,
h) a sequence at least 80, 85, 90, 95, 96, 97, 98 or 99% identical to the sequence of (g);
i) at least 6, 8, 10, 12, 15, 18, 20, 25, 50, 75, 90, 100, 120, 125, 130, 135, 140, 145, 150, 155 or 160 consecutive amino acids of the sequence of (g) or (h);
j) the sequence consisting of amino acids 1240 to 1270 of SEQ ID NO: 1;
k) a sequence at least 80, 85, 90, 95, 96, 97, 98 or 99% identical to the sequence of (j);
l) at least 6, 8, 10, 12, 15, 18, 20 or 25 consecutive amino acids of the sequence of (j) or (k); or
m) the sequence of (k) or (l), wherein said sequence comprises amino acids 1249 to 1252 of SEQ ID NO: 1.

These antibodies can recognize an epitope located within, or comprising at least one amino acid located within, the fragment comprising or consisting of any one of (a) to (m).

Preferably, said epitope is located within the fragment comprising or consisting of any one of (a) to (j).

Most preferably said epitope is located within the fragment comprising or consisting of any one of (a) to (f). Such antibodies are characterized in that they specifically bind to ATIP3, but not to other ATIP isoforms.

These antibodies can advantageously be used when selecting a patient to be treated by a polypeptide comprising ATIP3 or a biologically active fragment thereof, e.g. using the method described hereabove.

These antibodies can be polyclonal or monoclonal. When the antibodies are monoclonal, they can for example correspond to chimeric, humanized or fully human antibodies.

Methods for obtaining such antibodies are well known in the art. For example, polyclonal antibodies according to the invention can be obtained through immunization of a non-human mammal with said fragment comprising or consisting of any one of (a) to (j). Starting from the polyclonal antibodies, one can then obtain monoclonal antibodies using standard methods.

All references cited herein, including journal articles or abstracts, published patent applications, issued patents or any other references, are entirely incorporated by reference herein, including all data, tables, figures and text presented in the cited references.

Although having distinct meanings, the terms “comprising”, “having”, “containing’ and “consisting of” may be replaced with one another throughout the above description of the invention.

In the frame of the present description, all polypeptides, nucleic acids and antibodies may optionally be isolated and/or purified.

The invention will be further evaluated in view of the following examples and figures.

### BRIEF DESCRIPTION OF THE FIGURES

FIG. 1 shows that ATIP3 delays the time-course of metastatic progression.

FIG. 2 shows that ATIP3 reduces the number of metastatic foci (at day 17).

FIG. 3 shows that ATIP3 reduces the formation of breast cancer metastases.

FIG. 4 shows that ATIP3 inhibits cell migration (in Boyden chambers)

FIG. 5 shows that ATIP3 inhibits cell migration (assessed through a wound healing experiment). Metastatic triple negative breast cancer MDA-MB-231-derived D3H2LN cells (Calipers') stably expressing GFP or GFP-ATIP3 were allowed to migrate in a wound healing assay, and analyzed by time-lapse videomicroscopy (1 image every 5 minutes for 11 hours). The Y-axis shows the percentage of closure of the wound.

FIG. 6 represents a scheme of ATIP3a, on which the localization of the regions and consensus sequences identified by the inventors are shown.

FIG. 7 shows that ATIP3a, ATIP3b and a fragment of ATIP3 that consists of residues 410-874 of SEQ ID NO: 1 (referred to as ATIP3BD) inhibit cell proliferation and reduce cancer cell growth (in clonogenicity experiments).

FIG. 8 shows that ATIP3 sensitizes breast cancer cells to a chemotherapy with taxol. “MIT” stands for tetrazolium salt.

FIG. 9 shows that ATIP3 silencing decreases the sensitivity of breast cancer cells to a chemotherapy with taxol. “MIT” stands for tetrazolium salt.

FIG. 10 shows the effect of ATIP3 on the sensitivity of breast cancer cells to a chemotherapy with two different anti-mitotic agents, namely docetaxel (upper panel) and taxol (lower panel). The experiment was carried out with the MCF7 hormono-dependent breast cancer cell line. The cells were seeded at 15000 cells/cell on gold electrodes (Xcelligence technology, Roche, Germany) and let adhere for 18 hours before adding 50 nM of docetaxel or taxol. Cell impedance was monitored every 5 minutes for 96 hours. The results were normalized to cell index at the time of drug addition.

FIG. 11 shows the effect of ATIP3 on the sensitivity of breast cancer cells to a chemotherapy with taxol. The experiment was carried out as described in the legend of FIG. 10, except that the D3H2LN breast cancer cell line was used. This cell line origins from a triple-negative breast cancer.

FIG. 12 shows that ATIP3a, D2N and D2C_inhibit cell proliferation and reduce cancer cell growth (in clonogenicity experiments).

FIG. 13 shows the effect of minimal domains D2N, D2C and D2CC on breast cancer cells proliferation.

### BRIEF DESCRIPTION OF THE SEQUENCES

SEQ ID NO: 1 shows a full-length amino acid sequence of ATIP3 (isoform ATIP3a).

SEQ ID Nos. 2 and 3 shows the sequence of primers used in example 2.

SEQ ID NO: 4 shows the sequence of a siRNA used in the examples.

SEQ ID NO: 5 shows the sequence of penetratin.

SEQ ID NO: 6 shows a full-length amino acid sequence of ATIP3 (isoform ATIP3b).

SEQ ID Nos. 7 and 12 show the sequence of primers used in example 6.

### EXAMPLES

### Example 1

### Full-Length ATIP3 has an Anti-Metastatic Effect

It has been previously shown that full-length ATIP3 is a novel microtubule-associated protein with anti-mitotic effects in vitro and in vivo (Rodrigues-Ferreira et al., PLoS One. 2009; 4(10):e7239 2009).

This effect has been confirmed in vivo in mice models of metastatic breast cancer. In addition, an anti-metastatic effect has been found.

Briefly, metastatic triple negative breast cancer MDA-MB-231-derived D3H2LN cells (Calipers') expressing luciferase ±GFP-ATIP3 were injected into nude mice through an intra-cardiac injection. The mice were then analyzed through bioluminescence imaging (IVIS) following intra-peritoneal injection of luciferin.

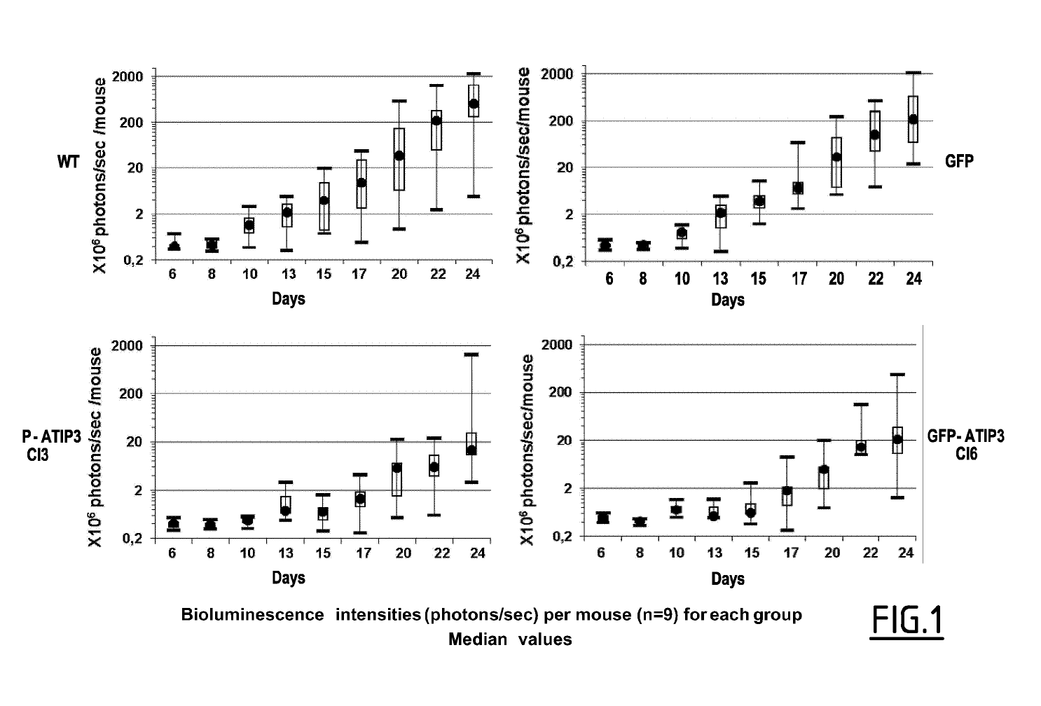

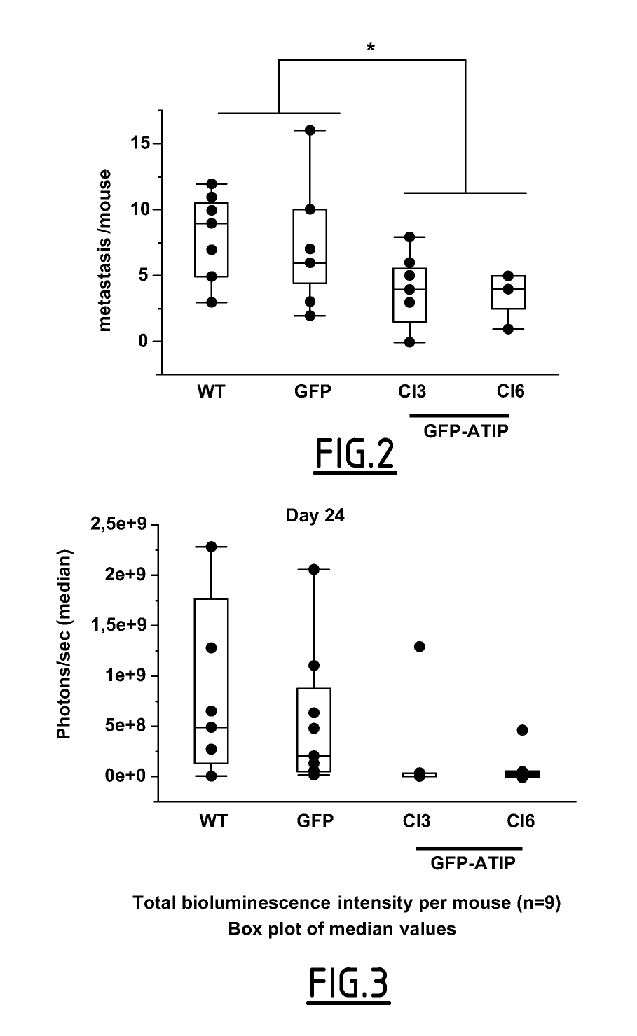

It was found that ATIP3:
delays the time-course of metastatic progression (FIG. 1);
reduces the number of breast cancer metastatic foci (FIG. 2);
reduces the size of breast cancer metastatic foci (data not shown);
reduces the number of mice with metastatic overgrowth (data not shown); and
reduces the formation of breast cancer metastases (FIG. 3);

### Example 2

### Identification of a Functional Domain of ATIP3

2.1. Identification of Different Domains of ATIP3

The inventors have found three domains within the ATIP3 primary sequence (NCBI Reference Sequence: NP_{—}001001924), said domains being characterized by different isoelectric points:
an acidic N-terminal region (residues 1-409 of SEQ ID NO: 1);
a basic central region (residues 410-874 of SEQ ID NO: 1); and
a neutral C-terminal region (residues 875-1270 of SEQ ID NO: 1).

The inventors have further identified two consensus sites for interaction with the end-binding protein (EB1), which is a crucial component of the growing ends of the microtubules (see e.g. Honnappa et al., 2009, Cell 138(2):366-76). These two consensus sites for binding with EB1 are:
a KNIP sequence (residues 462-465 of SEQ ID NO: 1), which is present in ATIP3a and ATIP3b. It is located within the basic central region described hereabove; and
a SAIP sequence (residues 1249-1252 of SEQ ID NO: 1), which is present in most ATIP isoforms.

Moreover, two major regions (predicted to be non-ordinary secondary structures) are believed to play a role in the interaction of ATIP3 with microtubules and the EB1 protein, namely the regions consisting of:
amino acids 333 to 580 of SEQ ID NO: 1 (247 residues). This region encompasses the KNIP sequence; and
amino acids 1240 to 1270 of SEQ ID NO: 1 (30 residues) This region encompasses the SAIP sequence.

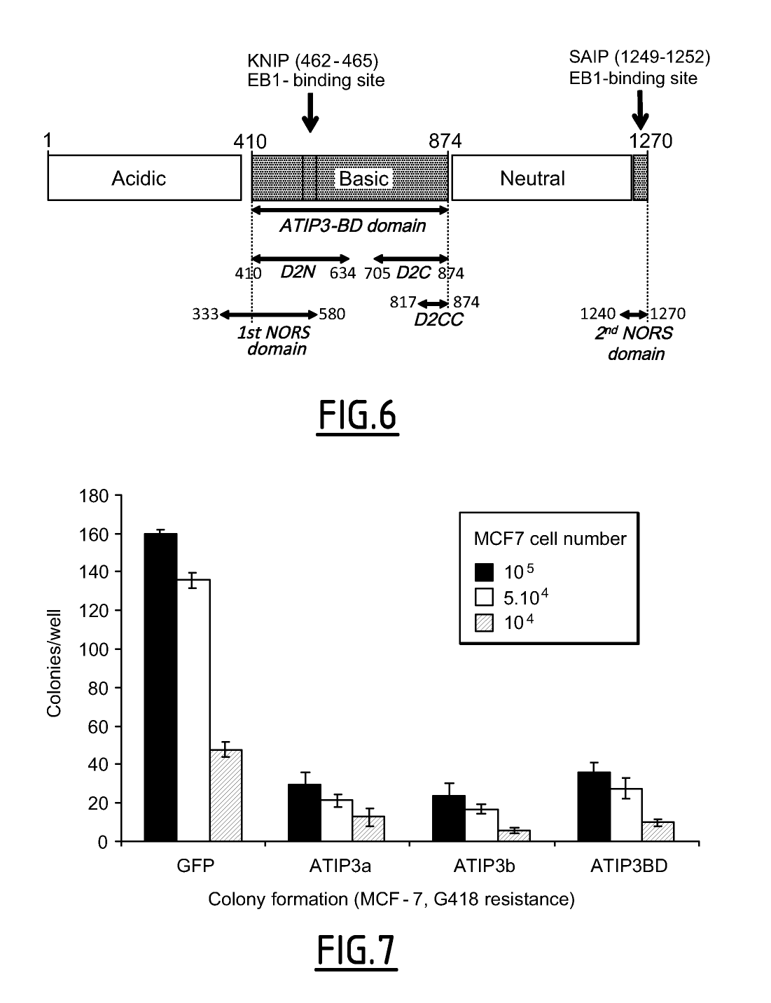

FIG. 6 depicts the localization of these regions and consensus sequences on ATIP3a.

2.2. Cloning of the Central Basic Domain of ATIP3 (ATIP3BD)

The basic central domain of ATIP3 (nucleotides 1228 to 2623 of the coding sequence, encoding residues 410-874 of SEQ ID NO: 1) was amplified by 35 rounds of PCR (annealing temperature 55° C.) using as a template the ATIP3 full length cDNA in pEGFP-C1 described in Rodrigues-Ferreira et al. (PLoS One. 2009; 4(10):e7239 2009) with the following primers:

Forward:
(SEQ ID NO: 2)
CCG CTC GAG GA CTG ACT TGG GAT GCA AAT GAT;
and
Reverse:
(SEQ ID NO: 3)
CGG GGT ACC TCA TGC ATT AAG AGC TGT AAA TAA.

The amplified 1.4 Kb PCR product was cleaved, purified and introduced between the KpnI-XhoI restriction sites of the pEGFP-C1 vector (Clontech), so that the resulting ATIP3 basic domain (ATIP3BD) is in frame with GFP. Expression of GFP-ATIP3BD fusion protein (which has an apparent molecular weight of 80 KDa) was confirmed by western blot using anti-GFP monoclonal antibodies.

2.3. ATIP3BD Interacts with Microtubules

The pEGFP-ATIP3BD plasmid was transiently transfected into human breast cancer MCF-7 cells. Localization of GFP-ATIP3BD fusion protein at the microtubule cytoskeleton was examined by immunofluorescence, and its ability to interact with stabilized microtubules was determined by co-sedimentation assay.

Immunofluorescence Studies

Exponentially growing human breast cancer MCF-7 cells plated on coverslips were transiently transfected (24 h) with pEGFP-ATIP3BD plasmid, and fixed in ice-cold methanol for 5 min prior to incubation for 1 h at room temperature with primary antibodies: anti-GFP monoclonal antibodies (Roche, diluted 1:200) and human anti-alpha-tubulin antibodies clone F2C (diluted 1:10), as described in Rodrigues-Ferreira et al. (PLoS One. 2009; 4(10):e7239 2009). After extensive washing, cells were incubated for 30 min at room temperature with appropriate secondary Cy-2-conjugated anti-human and/or Cy-3-conjugated anti-mouse antibodies (Jackson Laboratories, Interchim, France) diluted 1:500. Coverslips were mounted on glass slides using Glycergel Mounting Medium (DakoCytomation) and examined with a Leica TCS SP2 AOBS confocal microscope using appropriate filters. Image analysis was performed with NIH ImageJ software.

Microtubule Co-Sedimentation Assay

For microtubule cosedimentation assay, human breast cancer MCF-7 cells were transiently transfected (24 h) with pEGFP-ATIP3BD plasmid and incubated for 20 min at 4° C. in PEM buffer (100 mM PIPES, pH 6.9, 1 mM MgSO4, 1 mM EGTA), scraped and sonicated prior to centrifugation at 15000 rpm for 10 min, 4° C. Clarified samples were incubated with taxol (20 μM) in the presence of GTP (1 mM) and DTT (1 mM) for 45 min at 37° C. and were spun at 70 000 g for 30 min at 30° C. through a cushion buffer containing 40% glycerol, 20 μM taxol and 1 mM GTP. The supernatant (S) and pellet (P) fractions were collected separately and subsequently immunoblotted with anti-GFP (Roche) and anti-alpha-tubulin (Sigma) antibodies.

From these experiments, it was concluded that the ATIP3BD domain localizes at the microtubule cytoskeleton (immunofluoescence studies, data not shown), and co-sediments with stabilized microtubules (biochemical studies, data not shown).

Thus, it was shown that the ATIP3BD domain is sufficient to promote association of the protein with microtubules in intact cells

2.4. ATIP3BD Mimics the Entire ATIP3 Protein

Functional effects of GFP-ATIP3BD on breast cancer cell proliferation were analyzed by clonogenicity experiments.

Clonogenicity

For clonogenicity experiments, human breast cancer MCF-7 cells were transfected for 24 h with 0.2 μg pEGFP-C1 vector or 2 μg of pEGFP-ATIP3BD cDNA. Similar transfection efficiency (50%) was assessed by FACS analysis and cell viability was verified using trypan blue. Cells were then plated at various dilutions in 12 well plates and transfectants were selected by adding geneticin (G418, 1 mg/ml, Gibco) in fresh medium twice a week for 3 weeks. Resistant colonies were stained with 0.5% crystal violet (Sigma) and counted.

ATIP3BD was shown to mimic the inhibitory effects of the entire ATIP3a and ATIP3b proteins (see FIG. 7).

### Example 3

### ATIP3 Increases Sensitivity of Breast Cancer Cells to Taxol Treatment

Taxol (Paclitaxel) is a microtubule stabilizing agent that targets the mitotic spindle and is largely used in conventional chemotherapy of breast cancer, to kill tumor cells undergoing rapid division. Studies have been performed to evaluate whether ATIP3 may increase the effects of taxol on microtubule stability, and hence whether re-expressing ATIP3 may sensitize cancer cells to lower doses of taxol.

Experiments were performed on 3 independent clones of MCF-7 breast cancer cell lines stably expressing GFP-ATIP3 (namely HCl, HC6, HC7, described in Rodrigues-Ferreira et al., PlosOne 2009). Cells were seeded in quadruplicate in 96-well plates at the density of 5000 cells per well. The day after, cells were treated with increasing concentrations of taxol (Paclitaxel) for 48 h. Cell proliferation was then analyzed by incubation for 4 h at 37° C. with 1 mg/ml tetrazolium salt MTT (Sigma). Cleavage products (formazan) were solubilized with DMSO and optical density was measured at 560 nm, as recommended by the manufacturer.

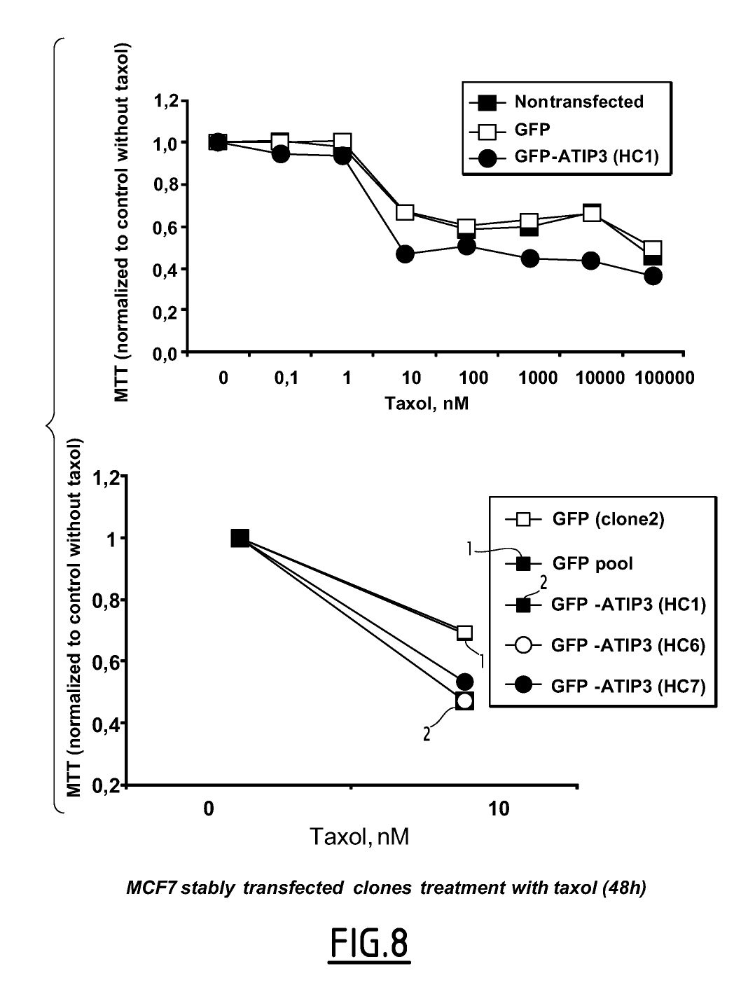

The results are shown on FIG. 8. In the presence of GFP-ATIP3, a lower dose of taxol (paclitaxel) (two-fold lower) was sufficient to reduce cell proliferation/viability to the same extent as GFP alone. Thus, re-expression of GFP-ATIP3 into breast cancer cells MCF-7 sensitizes cancer cells to the effect of taxol.

In another set of experiments, MDA-MB-468 breast cancer cells (that express endogenous ATIP3 proteins) were transfected with specific siRNA and the consequence of ATIP3 down-regulation on cancer cell sensitivity to taxol was evaluated using MTT (Sigma) as described above. Small interfering RNA (siRNA) was transfected at 50 nM for 72 h using Lipofectamine reagent 2000. Control siRNA duplex (non targeting pool #1), and specific MTUS1 siRNA#1 (on-target plus smart pool, NMO20749) and siRNA#2 (on-target plus siRNA duplex, sens strand: UGG CAG AGG UUU AAG GUU A; SEQ ID NO: 4) were purchased from Dharmacon (Chicago, Ill., USA). Silencing efficiency was measured by western blot and real time RT-PCR.

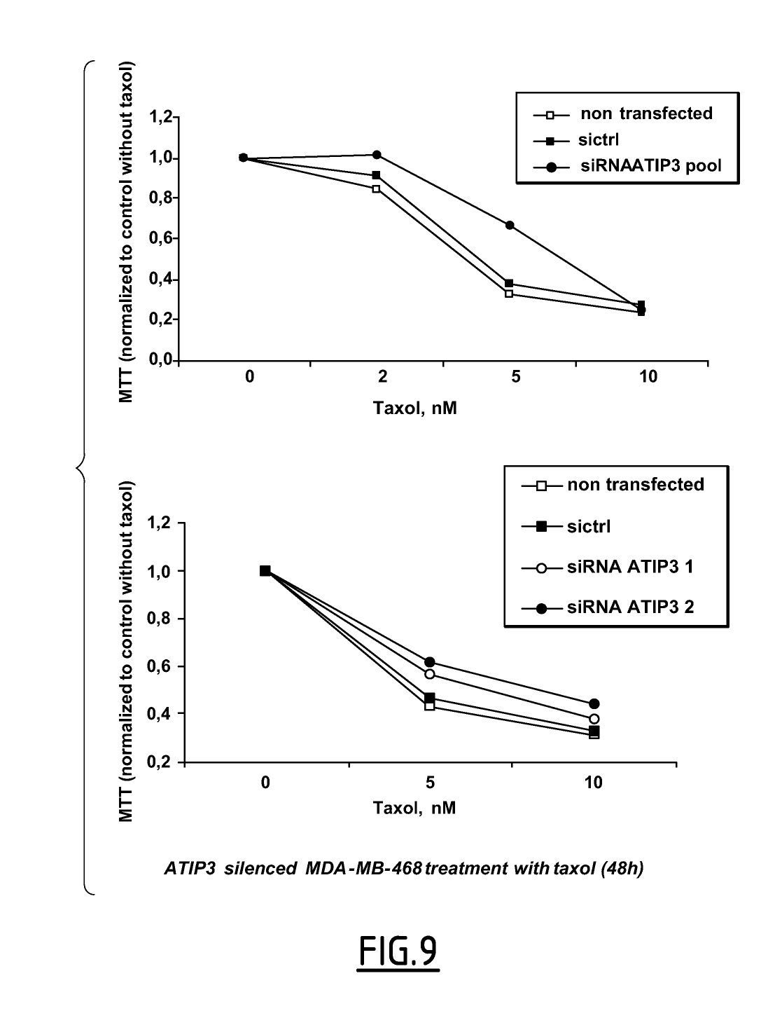

The results are shown on FIG. 9. It can be seen that silencing ATIP3 decreases the sensitivity of breast cancer cells to chemotherapy with Taxol, thereby confirming the effect of ATIP3 on sensitivity to the Taxol anti-mitotic agent.

In still another set of experiments, it was confirmed that ATIP3 sensitizes breast cancer cells to chemotherapy by studying a second anti-mitotic agent in addition to taxol, and by using another cell line. More specifically, breast cancer cells (MCF7 or triple negative metastatic D3H2LN) were seeded at 15000 cells/well on gold electrodes (Xcelligence technology, Roche) and let adhere for 18 hours before adding 50 nM taxotere (docetaxel) or taxol (paclitaxel). Cell impedance was monitored every 5 minutes for 96 hours. Results were normalized to cell index at the time of drug addition.

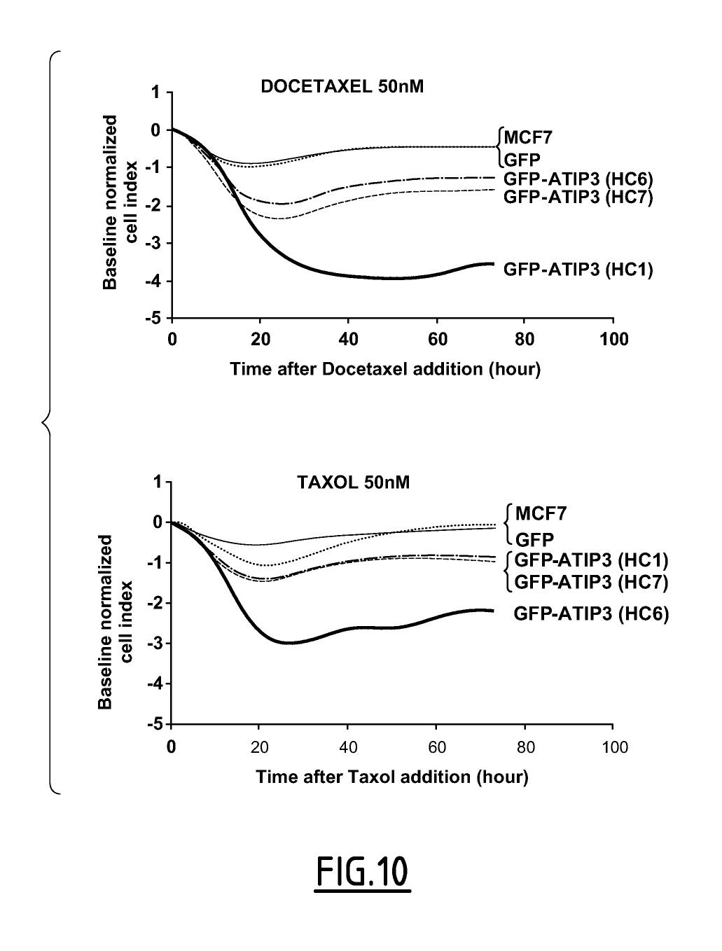

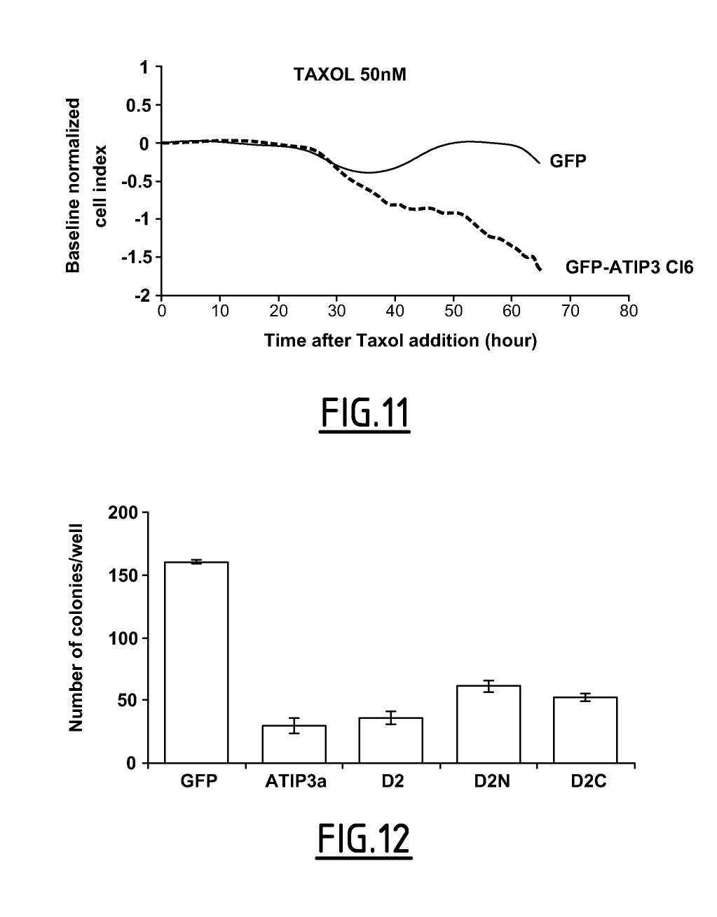

The results are shown on FIGS. 10 and 11. It was found that re-expression of GFP-ATIP3 into breast cancer cells sensitizes cancer cells not only to the effect of taxol, but also to the effect of docetaxel. In addition, it was found that this effect is not only seen in MCF7 hormono-dependent cells (FIG. 10) but also in triple negative metastatic cells (FIG. 11).

### Example 4

### Characterization of the Biological Activity of ATIP3

The capacity of ATIP3 to inhibit and/or reduce cell migration was assessed through Boyden chambers experiments and through wound healing experiments.

The experiments were performed on two independent clones (namely C13, C16) of metastatic breast cancer cells D3H2LN (derived from triple negative breast cancer cells MDA-MB-231, Calipers) stably expressing GFP-ATIP3, and compared to wild-type and GFP-expressing cell clones.

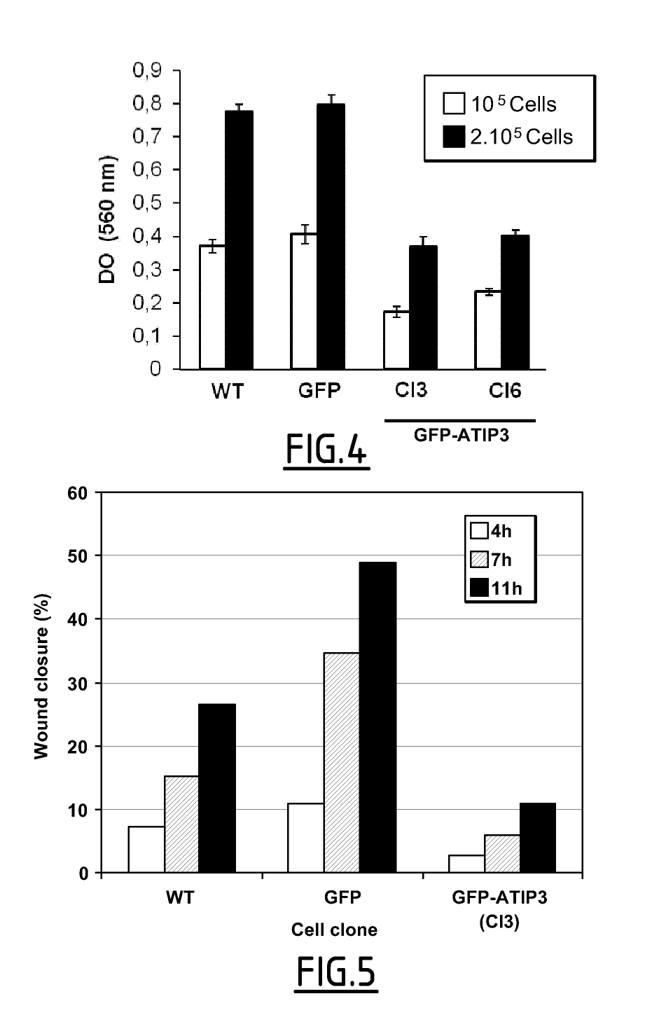

To measure cancer cell migration (Boyden chambers) in response to chimioattractant, cells were starved for 24 hours and seeded in quadruplicate at a density of 100000 or 200000 cells per well, on Boyden chambers containing transwell filters (diameter 6.5 mm, pore size 8 μm). Fetal calf serum (FCS) at 10% was added to the lower compartment, and cells were allowed to migrate for 18 hours, before the filter was fixed with ethanol. Cells having migrated underneath the filter were stained with 0.5% crystal violet (Sigma). After solubilization of crystal violet in DMSO, optical density was measured at 560 nm (see FIG. 4).

To measure cancer cell migration in wound healing experiments, cells were plated on IBIDI chambers at a density of 100.000 cells for 24 h to form a monolayer. The plastic insert of IBIDI chamber was then removed, leaving an empty space between the two borders, and cells were alllowed to migrate for 4 h, 7 h or 11 h in the presence of 10% FCS, before a picture was taken (phase contrast, magnification ×10). Closure of the wound was calculated using ImageJ, by measuring the area that remained empty between cells (see FIG. 5).

It was found that ATIP3 inhibits cell migration, as shown on FIGS. 4 and 5.

### Example 5

### Further Experiments

Fusion proteins between ATIP3 or ATIP3BD and penetratin are constructed. Penetratin is a 16 amino acids-long peptide (RQIKIWFQNRRMKWKK, SEQ ID NO: 5) able to cross the membranes, which can be used to transduce proteins inside the cell (Prochiantz, 2008, Adv Drug Deliv Rev. 60:448-51). The fusion protein is tagged with a (6×His) epitope in a prokaryotic expression vector, to allow production in E. coli and purification on nickel columns. The resulting proteins are termed ATIP3-pen or ATIP3BD-pen.

The functional effects of increasing doses of ATIP3-pen or ATIP3BD-pen, following transduction into breast cancer cells (MDA-MB-231 and MCF-7, which do not express ATIP3), is evaluated in the presence or in the absence of increasing doses of taxanes.

The consequence of transducing ATIP3-pen or ATIP3BD-pen into human endothelial cells (HUVEC), as a model of non-tumoral cells of the stroma, is studied.

The effects of ATIP3-pen are investigated in vivo. Increasing quantities of ATIP3-pen fusion protein is administered into the bloodstream of mice, at different times of development of a primary tumor (subcutaneous injection) or distant metastasis (intracardiac injection).

### Example 6

### Identification of Minimal Functional Domain of ATIP3 which can Mimic the Entire ATIP3 Protein

ATIP3BD has been subdivided into several domains (D2N, D2C and D2CC encompassing amino-acid positions 410-634, 705-874 and 817-874 of SEQ ID NO: 1, respectively) and the ability of each of these domains to associate with microtubule and to reduce breast cancer cell proliferation was analyzed following their expression into breast cancer cells.

6.1 Cloning of Minimal Functional Domains of ATIP3

cDNA fragments encoding domains D2N, D2C and D2CC were amplified by 35 rounds of PCR (annealing temperature 55° C.) using as a template the full-length ATIP3 cDNA fragment (SEQ ID NO:1) and the following oligonucleotides:

D2N (F) (SEQ ID NO: 7):
5′ CCG CT CGA GGA CTG ACT TGG GAT GCA AAT
GAT -3′
D2N (R) (SEQ ID NO: 8):
5′- CGG GGT ACC TCA AAA CAA CGC AGA AAC GGA
CCC GGT-3′
D2C (F) (SEQ ID NO: 9):
5′ CC GCT CGA GCA TAT GGT TCT CTG TTT TTG GGC
TCT 3′
D2C (R) (SEQ ID NO: 10):
5′- CGG GGT ACC TCA TGC ATT AAG AGC TGT AAA
TAA -3′
D2CC (F) (SEQ ID NO: 11):
5′- CC GCT CGA GGA TCT GGT AAT GCC GCT
GTC ATC -3′
D2CC (R) (SEQ ID NO: 12):
5′- CGG GGT ACC TCA TGC ATT AAG AGC TGT
AAA TAA -3′

Amplified PCR products (676 bp, 511 bp and 175 bp for D2N, D2C and D2CC, respectively) were subcloned into the XhoI/KpnI restriction sites of the pEGFP-C1 expression vector (Clontech) so that the translated product is in frame with GFP.

Expression of corresponding GFP-D2N, GFP-D2C and GFP-D2CC fusion proteins in human cells (breast cancer MCF7 and non-tumoral RPE1) was confirmed by western blot of total cell lysates using anti-GFP monoclonal antibodies (ROCHE). Polypeptides of expected molecular weights (50 KDa for D2N, 42 KDa for D2C and 31 KDa for D2CC) were revealed.

6.2 Functional Analysis of Minimal Domains D2N, D2C and D2CC

Intracellular Localization

Immunofluorescence study was performed as example 2. Briefly, Human retinal pigment epithelial (RPE1) cells were transfected for 24 hrs with GFP-D2N, GFP-D2C or GFP-D2CC constructs prior to fixation in ice-cold methanol and incubation with anti-GFP antibodies as described (page 21, lines 1-9). Immunofluorescence assays (data not shown) clearly show that GFP-D2N and GFP-D2C both associate with the microtubule cytoskeleton whereas GFP-D2CC remains diffuse in the cytosol.

Clonogenicity

The ability of human MCF7 breast cancer cells to form colonies following expression of minimal domains D2N and D2C was analyzed by clonogenicity experiments as described before (see example 2).

FIG. 12 indicates that expression of GFP-D2N and GFP-D2C strongly reduces the number of G418-resistant colonies as compared to GFP. Inhibitory effects of D2N and D2C reach 71% and 78%, respectively, of that of GFP-ATIP3.

Xcelligence

Stable clones of MCF7 breast cancer cells expressing moderate levels of GFP-D2N (clones D2N6 and D2N8), GFP-D2C (clone D2C6 and a pool of positive cells) and GFP-D2CC (clone Cl3) have been selected in G418 (1 mg/ml)-containing medium for 4 weeks. Detectable expression of a single polypeptide of expected molecular weight in each case was confirmed by western blotting using anti-GFP antibodies.

Cell proliferation was measured using the Xcelligence technology (Roche). Briefly, cell clones were seeded at 15000 cells/well on 96 wells containing gold electrodes and cellular impedance was monitored in real-time for 40 hours.

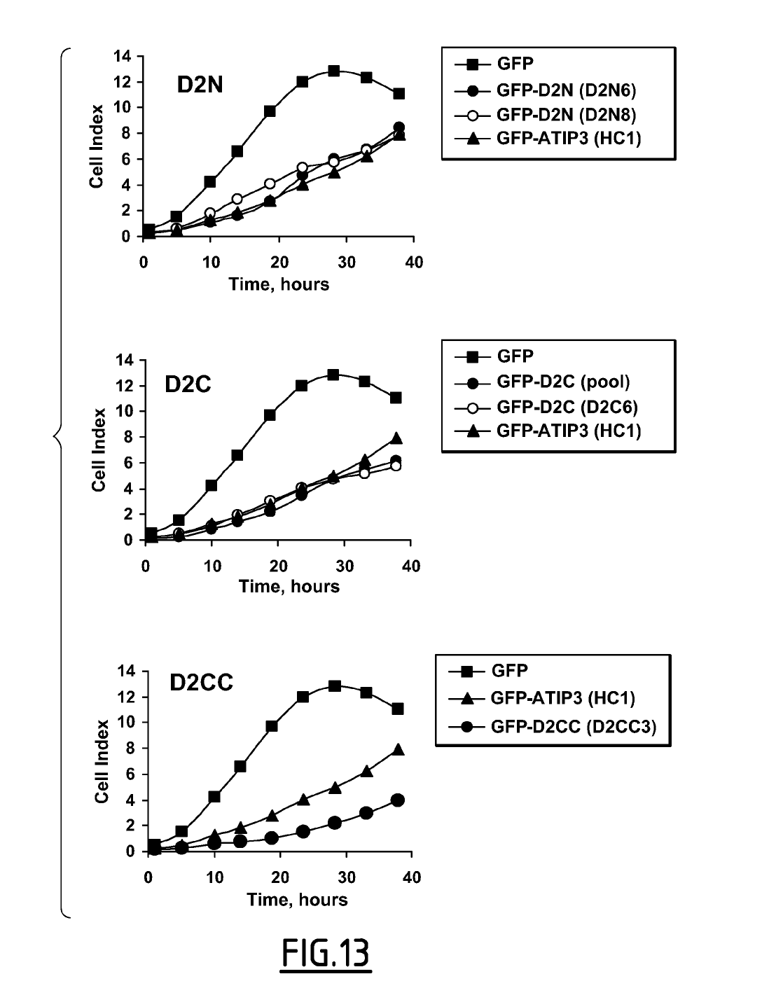

FIG. 13 shows that for each independent clone expressing either GFP-D2N (clones D2N6, D2N8), or GFP-D2C (D2C6 and pool), the proliferation rate was similar to that of clone HCl expressing GFP-ATIP3 and strongly reduced as compared to GFP-transfected cells.

Of interest, proliferation of clone D2CC3 (stably expressing the GFP-D2CC domain) was significantly reduced (by a factor of 2) as compared to that of clone HCl, indicating high anti-proliferative effects of this 58 amino-acids polypeptide upon expression in breast cancer cells.

The two minimal functional domains of ATIP3, namely D2N (224 aa) and D2C (170 aa) are able to associate with microtubules and reduce cell proliferation to the same extent as full-length ATIP3. These are very good candidates for anti-cancer therapies.

The D2CC domain (58 aa) is of interest and exhibits high anti-proliferative properties (2 fold-increased as compared to ATIP3) although it does not associate with microtubules. This domain thus appears as a promising potential therapeutic target.

1. A polypeptide comprising a biologically active fragment of at most 500 consecutive amino acids of Angiotensin-II type 2-receptor interacting protein 3 (ATIP3), wherein said fragment comprises:
a) the sequence consisting of amino acids 410 to 874 of SEQ ID NO: 1;
b) the sequence consisting of amino acids 410 to 820 of SEQ ID NO: 6;
c) the sequence consisting of amino acids 333 to 580 of SEQ ID NO: 1;
d) a sequence at least 80% identical to the sequence of (a), (b) or (c);
e) at least six consecutive amino acids of the sequence of (a), (b) or (c);
f) the sequence of (d) or (e), wherein said sequence comprises amino acids 462 to 465 of SEQ ID NO: 1 or SEQ ID NO: 6 or amino acids 410 to 634 or 705 to 874 or 817 to 874 of SEQ ID NO: 1;
g) the sequence consisting of amino acids 410 to 634 or 705 to 874 or 817 to 874 of SEQ ID NO: 1;
h) a sequence at least 80% identical to the sequence of (g); or
i) at least six consecutive amino acids of the sequence of (g) or (h).

2. The polypeptide according to claim 1, wherein said polypeptide further comprises penetratin.

3-13. (canceled)

14. An in vitro method for selecting a patient suffering from a cancer to be treated by a polypeptide comprising Angiotensin-II type 2-receptor interacting protein 3 (ATIP3) or a biologically active fragment thereof, or by said polypeptide in combination with a chemotherapeutic drug that is an anti-mitotic agent, wherein said method comprises the steps of:
a) providing a biological sample comprising cells from said cancer;
b) determining whether:
said cancer is a triple-negative breast cancer; and/or
ATIP3 is expressed in said biological sample; and
c) selecting said patient if he suffers from a triple-negative breast cancer, and/or if ATIP3 is expressed at a significantly lower level in said biological sample than in a control sample.

15. An antibody that specifically binds to a polypeptide comprising a biologically active fragment of at most 500 consecutive amino acids of Angiotensin-II type 2-receptor interacting protein 3 (ATIP3), wherein said fragment comprises:
a) the sequence consisting of amino acids 410 to 874 of SEQ ID NO: 1;
b) the sequence consisting of amino acids 410 to 820 of SEQ ID NO: 6;
c) the sequence consisting of amino acids 333 to 580 of SEQ ID NO: 1;
d) the sequence consisting of amino acids 410 to 634 or 705 to 874 or 817 to 874 of SEQ ID NO: 1,
e) a sequence at least 80% identical to the sequence of (a), (b) (c) or (d);
f) at least six consecutive amino acids of the sequence of (a), (b), (c), (d) or (e);
g) the sequence of (e) or (f), wherein said sequence comprises amino acids 462 to 465 of SEQ ID NO: 1 or SEQ ID NO: 6 or amino acids 410 to 634 or 705 to 874 or 817 to 874 of SEQ ID NO: 1.

16. A method of treating an individual in need thereof, said method comprising the step of administering to said individual a polypeptide comprising a biologically active fragment of at most 500 consecutive amino acids of Angiotensin-II type 2-receptor interacting protein 3 (ATIP3), wherein said fragment comprises:
a) the sequence consisting of amino acids 410 to 874 of SEQ ID NO: 1;
b) the sequence consisting of amino acids 410 to 820 of SEQ ID NO: 6;
c) the sequence consisting of amino acids 333 to 580 of SEQ ID NO: 1;
d) a sequence at least 80% identical to the sequence of (a), (b) or (c);
e) at least six consecutive amino acids of the sequence of (a), (b) or (c);
f) the sequence of (d) or (e), wherein said sequence comprises amino acids 462 to 465 of SEQ ID NO: 1 or SEQ ID NO: 6 or amino acids 410 to 634 or 705 to 874 or 817 to 874 of SEQ ID NO: 1;
g) the sequence consisting of amino acids 410 to 634 or 705 to 874 or 817 to 874 of SEQ ID NO: 1;
h) a sequence at least 80% identical to the sequence of (g); or
i) at least six consecutive amino acids of the sequence of (g) or (h).

17. The method according to claim 16 wherein said individual suffers from cancer.

18. The method according to claim 17 wherein said cancer is selected from the group consisting of breast cancer, pancreatic cancer, ovary cancer, head-and-neck cancer, colon cancer, colorectal cancer, liver cancer, prostate cancer, bladder cancer, stomach cancer and non-small-cell lung carcinoma.

19. The method according to claim 18, wherein said cancer is a cancer of high histological grade and/or a metastatic cancer.

20. The method according to claim 18, wherein said cancer is a triple-negative breast cancer.

21. A method of treating an individual suffering from cancer, said method comprising the step of administering simultaneously or sequentially to said individual an effective amount of:
i. a polypeptide comprising Angiotensin-II type 2-receptor interacting protein 3 (ATIP3) or a biologically active fragment thereof; and
ii. a chemotherapeutic drug that is an anti-mitotic agent.

22. The method according to claim 21, wherein said cancer is an ATIP3 under-expressing cancer.

23. The method according to claim 21, wherein said cancer is selected from the group consisting of breast cancer, pancreatic cancer, ovary cancer, head-and-neck cancer, colon cancer, colorectal cancer, liver cancer, prostate cancer, bladder cancer, stomach cancer and non-small-cell lung carcinoma.

24. The method according to claim 21, wherein said chemotherapeutic drug is a taxane preferably, paclitaxel or docetaxel.

25. The method according to claim 21, wherein said chemotherapeutic drug is administered at a lower dose than the dose recommended when said drug is administered without said polypeptide comprising ATIP3 or a biologically active fragment thereof.

26. The method according to claim 21, wherein said polypeptide comprising ATIP3 or a biologically active fragment thereof is a polypeptide comprising a biologically active fragment of at most 500 consecutive amino acids of ATIP3, wherein said fragment comprises:
a) the sequence consisting of amino acids 410 to 874 of SEQ ID NO: 1;
b) the sequence consisting of amino acids 410 to 820 of SEQ ID NO: 6;
c) the sequence consisting of amino acids 333 to 580 of SEQ ID NO: 1;
d) a sequence at least 80% identical to the sequence of (a), (b) or (c);
e) at least six consecutive amino acids of the sequence of (a), (b) or (c);
f) the sequence of (d) or (e), wherein said sequence comprises amino acids 462 to 465 of SEQ ID NO: 1 or SEQ ID NO: 6 or amino acids 410 to 634 or 705 to 874 or 817 to 874 of SEQ ID NO: 1;
g) the sequence consisting of amino acids 410 to 634 or 705 to 874 or 817 to 874 of SEQ ID NO: 1;
h) a sequence at least 80% identical to the sequence of (g); or
i) at least six consecutive amino acids of the sequence of (g) or (h).

27. The method according to claim 21, wherein said cancer is a cancer of high histological grade, a metastatic cancer or a triple-negative breast cancer.

28. A method of treating an individual suffering from ATIP3 under-expressing cancer, for sustaining drug effect or for increasing, restoring or enhancing sensitivity of a patient suffering from cancer to a chemotherapeutic drug that is an anti-mitotic agent wherein said method comprises the step of administering an effective amount of a polypeptide comprising Angiotensin-II type 2-receptor interacting protein 3 (ATIP3) or a biologically active fragment thereof to said individual.

29. The method according to claim 28, wherein said polypeptide comprising ATIP3 or a biologically active fragment thereof is a polypeptide comprising a biologically active fragment of at most 500 consecutive amino acids of ATIP3, wherein said fragment comprises:
a) the sequence consisting of amino acids 410 to 874 of SEQ ID NO: 1;
b) the sequence consisting of amino acids 410 to 820 of SEQ ID NO: 6;
c) the sequence consisting of amino acids 333 to 580 of SEQ ID NO: 1;
d) a sequence at least 80% identical to the sequence of (a), (b) or (c);
e) at least six consecutive amino acids of the sequence of (a), (b) or (c);
f) the sequence of (d) or (e), wherein said sequence comprises amino acids 462 to 465 of SEQ ID NO: 1 or SEQ ID NO: 6 or amino acids 410 to 634 or 705 to 874 or 817 to 874 of SEQ ID NO: 1;
g) the sequence consisting of amino acids 410 to 634 or 705 to 874 or 817 to 874 of SEQ ID NO: 1;
h) a sequence at least 80% identical to the sequence of (g); or
i) at least six consecutive amino acids of the sequence of (g) or (h).

30. The method according to claim 28, wherein said cancer is selected from the group consisting of breast cancer, pancreatic cancer, ovary cancer, head-and-neck cancer, colon cancer, colorectal cancer, liver cancer, prostate cancer, bladder cancer, stomach cancer and non-small-cell lung carcinoma.

31. The method according to claim 28, wherein said cancer is a cancer of high histological grade and/or a metastatic cancer and/or a triple-negative breast cancer.
